(19) United States
(12) Patent Application Publication
(10) Pub. No.: US 2016/0151435 A1
(43) Pub. Date: Jun. 2, 2016

(54) PHARMACEUTICAL COMPOSITION ADJUVANT TO CHEMOTHERAPY DRUGS AND APPLICATIONS THEREOF

(71) Applicant: BALAY BIOTECHNOLOGY CORPORATION, TAIPEI CITY (TW)

(72) Inventors: FENG-YUE WENG, TAIPEI CITY (TW); WEN-HUANG LIU, HSINCHU COUNTY (TW); CHIEN-LIANG KUO, CHANGHUA COUNTY (TW); CHIA-CHI LIN, TAIPEI CITY (TW)

(21) Appl. No.: 14/947,147

(22) Filed: Nov. 20, 2015

(30) Foreign Application Priority Data
Nov. 28, 2014 (TW) 103141511

(51) Int. Cl. A61K 36/07; A61K 31/36; A61K 31/575; A61K 31/7068; A61K 33/24

(52) U.S. Cl. CPC A61K 36/07; A61K 31/7068; A61K 33/24; A61K 31/575; A61K 31/36

(57) ABSTRACT

The present invention discloses a pharmaceutical composition and applications thereof. The pharmaceutical composition comprises an extract of Antrodia cinnamomea fruiting bodies and a pharmaceutical carrier. The extract is fabricated via soaking the powder of Antrodia cinnamomea fruiting bodies in hot water and then undertaking extraction with a low-polarity solvent to acquire the extract from the soaked powder. The pharmaceutical composition can function as an adjuvant drug of chemotherapy drugs to enhance the treatment effect of the chemotherapy drugs cisplatin and gemcitabine and relieve the chemotherapy drug-induced side-effects of hair loss, muscle degradation and atrophy, gastrointestinal lesion, nephritis and renal injury.

This application claims priority for Taiwan patent application no. 103141511 filed on Nov. 28, 2014, the content of which is incorporated by reference in its entirely.

### BACKGROUND OF THE INVENTION

1. Field of the Invention

The present invention relates to an extract of Antrodia cinnamomea and applications thereof, particularly to a pharmaceutical composition adjuvant to chemotherapy drugs and applications thereof.

2. Description of the Related Art

Antrodia cinnamomea is an endemic mushroom growing on Cinnamomum kanehirai Hayata only appearing in middle or high-altitude mountains of Taiwan. The aboriginals regard Antrodia cinnamomea as a precious mushroom able to protect livers and metabolize alcohol. The research reports from 1995 show that the Antrodia cinnamomea fruiting body (ACF) has many pharmaceutical effects, including anti-inflammation, immunity enhancement, physical strength improvement, antivirus, antioxidation, anticancer, and liver protection. Among them, several papers used in vivo models to verify the effects of the ACF on cancers, including liver cancer, lung cancer, colon cancer, and ovary cancer, and proved that the ethanol extract of ACF has a significant anticancer effect. However, logs of Cinnamomum kanehirai Hayata are hard to acquire. Thus, the cost for cultivating ACF is very high, and ACF are hard to massively supply. Further, ACF are likely to be polluted by harmful microorganisms and heavy metals. These abovementioned factors seriously delay the progress of developing ACF into new botanical drugs. Therefore, a critical technology to mass-produce the stable and high-quality raw material of ACF, free of heterogeneous population and heavy metals, is critical to the ACF industry development.

Mounting scientific evidences indicated that the normal human cells can acquire energy from metabolism of carbohydrate, protein and fat. However, cancer cells acquire energy merely from glucose and grow faster than normal cells. Therefore, a large part of glucose will be consumed by the cancer cells in the cancer patients. Further, growth of cancer cells will affect biosynthesis of protein, cause biodegradation and atrophy of muscle tissues, and weaken the gestation, olfaction and appetite of patients. Finally, most of cancer patients will suffer from cachexia syndromes, such as malnutrition, organ failure, and immunodeficiency.

Lung cancer is one of the cancers having the highest prevalence and mortality. Lung cancer is mainly divided into small cell lung cancer (having an incidence rate of about 16.8%) and non-small cell lung cancer (having an incidence cancer of about 80.4%). Lung cancer is usually treated with surgery, radiotherapy and medical therapy (including chemotherapy and targeted drugs). Chemotherapy has been one of the primary choices to treat lung cancer. At present, the first-line chemotherapy drugs are gemcitabine and cisplatin. The effect of the combination of the first-line chemotherapy drugs can persist for over half a year in average. Then, if the cancer is not controlled by the first-line chemotherapy drugs, a second-line chemotherapy drug (a single type of drug) will be applied to the patients, such as gemcitabine or Taxol injection, or oral/intravenous Vinorelbine. Although chemotherapy can successfully kill cancer cells, it also kills normal cells unselectively. Chemotherapy drugs kill abnormal and normal cells simultaneously and usually seriously harm the immunity system and hematopoietic system of patients. Chemotherapy drugs particularly affect the fast-growing cells although they are normal, including epithelial cells of oral cavities and gastrointestinal tracts, hematopoietic cells of bone marrows, and hair follicle cells. Therefore, chemotherapy drugs usually cause serious side-effects to patients, including anergy, nausea, vomiting, blood cell decrease, alopecia, oral ulcer and pain.

Considering that the side-effects of chemotherapy cause discomfort to cancer patients and retard the cancer patients from completing the chemotherapy, complementary treatments intervene in chemotherapy to relieve the discomfort caused by chemotherapy so that cancer patients can complete chemotherapy.

Recently, the chemotherapy drugs have encountered many bottlenecks in development process and even caused serious side-effects in clinical application. Thus, the related fields gradually turn their attention to developing botanical chemo-adjuvant therapy drugs. There have been some researches about the effects of ACF in relieving the cisplatin-induced hepatotoxicity and inflammatory intestinal diseases, the intestinal cancer-induced cachexia of mice, and the effect of ACF functioning as an adjuvant drug of breast cancer. These researches prove that ACF can function as an adjuvant drug for chemotherapy, enhancing the effect of chemotherapy and reducing the side-effects of chemotherapy.

### SUMMARY OF THE INVENTION

The primary objective of the present invention is to provide a pharmaceutical composition adjuvant to chemotherapy drugs and applications thereof, wherein the extract of ACF is used to combine with chemotherapy drugs cisplatin and gemcitabine for improving the effect of cancer treatment and relieve the syndromes of cancers and the side-effects of chemotherapy, such as alopecia, degradation and atrophy of muscle, and inflammation/lesion of gastrointestinal tracts and kidneys.

To achieve the abovementioned objective, the present invention proposes a pharmaceutical composition, which comprises an effective dosage of an extract of ACF and a pharmaceutical carrier, and which is used as an adjuvant to chemotherapy drugs to enhance the cancer treatment effect and relieve the side-effects of the chemotherapy drugs, wherein the chemotherapy drugs are cisplatin and gemcitabine.

In one embodiment, the extract of ACF comprises antcin K, antcin C, antcin H and derivatives thereof, which are expressed as Formula (I):

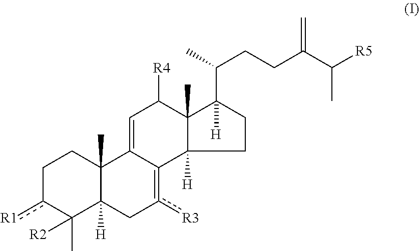

wherein
R1 is —OH, ═O, a —O-glycosyl group (a monosaccharide, disaccharide, or polysaccharide), or a C₁₋₃ ester group;
R2 is —H, —OH, a —O-glycosyl group (a monosaccharide, disaccharide, or polysaccharide), or a C₁₋₃ ester group;
R3 is —OH, ═O, a —O-glycosyl group (a monosaccharide, disaccharide, or polysaccharide), or a C₁₋₃ ester group;
R4 is —H, —OH, a —O-glycosyl group (a monosaccharide, disaccharide, or polysaccharide), or a C₁₋₃ ester group; and
R5 is a C₁₋₃ carboxyl group or a C₁₋₃ ester group.

In one embodiment, the extract of ACF also comprises dehydrosulphurenic acid, dehydroeburicoic acid and derivatives thereof, which are expressed as Formula (II):

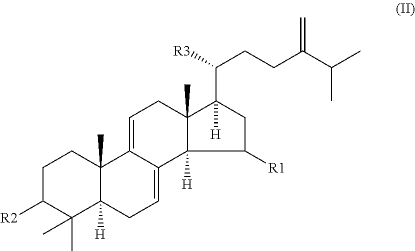

wherein
R1 is —H, —OH, a —O-glycosyl group (a monosaccharide, disaccharide, or polysaccharide), or a C₁₋₃ ester group;
R2 is —OH, a —O-glycosyl group (a monosaccharide, disaccharide, or polysaccharide), or a C₁₋₃ ester group; and
R3 is a C₁₋₃ carboxyl group or a C₁₋₃ ester group.

In one embodiment, the extract of ACF further comprises antcin B, antcin A and derivatives thereof, which are expressed as Formula (III):

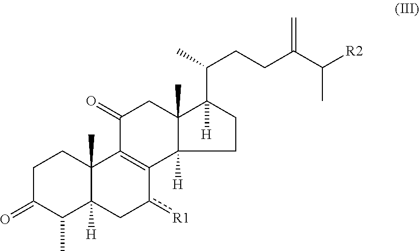

wherein
R1 is —H or ═O,
R2 is a C₁₋₃carboxyl group or a C₁₋₃ ester group.

In one embodiment, the extract of ACF further comprises 4,7-dimethoxy-5-methyl-1,3-benzodioxole (DMB) and derivatives thereof, which are expressed as Formula (IV):

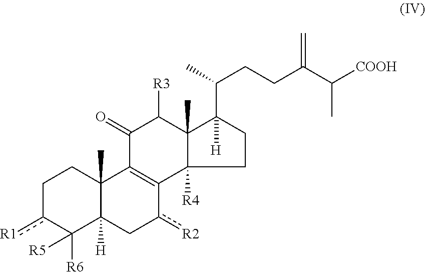

wherein
R1 is —OH, ═O, a —O-glycosyl group (a monosaccharide, disaccharide, or polysaccharide), or a C₁₋₃ ester group;
R2 is —H, —OH, ═O, a —O-glycosyl group (a monosaccharide, disaccharide, or polysaccharide), or a C₁₋₃ ester group;
R3 is —H, —OH, a —O-glycosyl group (a monosaccharide, disaccharide, or polysaccharide), or a C₁₋₃ ester group;
R4 is —H or a methyl group;
R5 is —H, —OH, a methyl group, a —O-glycosyl group (a monosaccharide, disaccharide, or polysaccharide), or a C₁₋₃ ester group; and R6 is —H or a methyl group.

The present invention also proposes an application of a pharmaceutical composition, wherein the pharmaceutical composition includes an effective dosage of an extract of ACF and a pharmaceutical carrier, and wherein the pharmaceutical composition is used as an adjuvant to chemotherapy drugs for improving the cancer treatment effect of the chemotherapy drugs and relieve the syndromes of cancers and the side-effects of the chemotherapy drugs, wherein the chemotherapy drugs are cisplatin and gemcitabine. In one embodiment, the effective dosage of the extract of ACF is 5.06-18.75 mg/kg.

It is proved by in vivo experiments: the lung weight of the group using a combination of the extract of ACF of the present invention, cisplatin and gemcitabine is significantly lighter than that of the lung cancer group by as much as 69.4% (p<0.005) significantly lighter than that of the group merely using the chemotherapy drugs by 30.6% (p<0.01); the number of lung cancer tubercles of the group using the combination is less than that of the lung cancer group by 55.4%; as to the improvement on degradation and atrophy of muscle, the weight of the thigh muscle of the group of mice using the combination is heavier than that of the group merely using the chemotherapy drugs by 42% (p<0.01); the pharmaceutical composition of the present invention can obviously inhibit the enzymatic activities of proteasomes in the muscle of the mice using the chemotherapy drugs cisplatin and gemcitabine, wherein the enzymatic activities of chymotrypsin, trypsin and caspase are respectively inhibited by 28% (p<0.01), 26.1% (p<0.01), and 11.1%; as to improving gastrointestinal lesion, the extract of ACF can greatly assuage the side-effects of intestinal villus damage and gastric ulcer caused by the chemotherapy drugs (cisplatin and gemcitabine) and can also improve the enzymatic activities of leucine aminopeptidase (LAP), lipase (LIP) and amylase in small intestines; as to the improvement on inflammation and damage of kidneys, the extract of ACF can effectively lower the post-chemotherapy nephritis indexes, such as creatinine, blood urea nitrogen (BUN), and serum albumin.

Therefore, the present invention has proved that the extract of ACF can be an adjuvant to chemotherapy drugs. The pharmaceutical composition can be fabricated into various forms of drugs to enhance the effect of cancer therapy and relieve the side-effects of chemotherapy.

Below, embodiments are described in detail to make easily understood the objectives, technical contents, characteristics and accomplishments of the present invention.

### BRIEF DESCRIPTION OF THE DRAWINGS

FIG. 1 shows a flowchart of a method for fabricating an extract of ACF of a pharmaceutical composition adjuvant to chemotherapy drugs according to one embodiment of the present invention;

FIG. 2 shows a HPLC spectrum of an extract of ACF according to one embodiment of the present invention;

FIG. 3 shows the effect of the extract of ACF in improving the chemotherapy drug-induced body weight loss of the experimental mice according to one embodiment of the present;

FIG. 4 shows the effect of the extract of ACF in improving the chemotherapy drug-induced intake decrease of the experimental mice according to one embodiment of the present invention, wherein (a) is the Normal group, (b) the Cancer group, (c) the CGC group, (d) the CGCA group, and (e) the CA group;

FIG. 5 shows the effect of the extract of ACF in reducing the lung weight of the lung cancer mice according to one embodiment of the present invention, wherein (a) is the Normal group, (b) the Cancer group, (c) the CGC group, (d) the CGCA group, and (e) the CA group;

FIG. 6 shows the effect of the extract of ACF in reducing the number of lung cancer tubercles of the lung cancer mice according to one embodiment of the present invention, wherein (a) is the Normal group, (b) the Cancer group, (c) the CGC group, (d) the CGCA group, and (e) the CA group;

FIG. 7 shows the effect of the extract of ACF in improving the chemotherapy drug-induced muscle degradation and atrophy of the experimental mice according to one embodiment of the present invention, wherein (a) is the Normal group, (b) the Cancer group, (c) the CGC group, (d) the CGCA group, and (e) the CA group;

FIG. 8 shows the effect of the extract of ACF in inhibiting the activities of the proteasomes in the muscle of the experimental mice using the chemotherapy drugs cisplatin and gemcitabine according to one embodiment of the present invention;

FIG. 9a and FIG. 9b respectively show the effects of the extract of ACF in inhibiting the expression of myostatin and encouraging the expression of IGF-1 in muscle according to one embodiment of the present invention, wherein (a) is the Normal group, (b) the Cancer group, (c) the CGC group, (d) the CGCA group, and (e) the CA group;

FIGS. 10a-10c respectively show the effects of the extract of ACF in inhibiting the chemotherapy drug-induced increase of blood inflammatory factors according to one embodiment of the present invention, wherein (a) is the Normal group, (b) the Cancer group, (c) the CGC group, (d) the CGCA group, and (e) the CA group;

FIGS. 11a-11c respectively show the effects of the extract of ACF in improving the post-chemotherapy expressions of the enzymes in the small intestine according to one embodiment of the present invention, wherein (a) is the Normal group, (b) the Cancer group, (c) the CGC group, (d) the CGCA group, and (e) the CA group;

FIGS. 12a-12c respectively show the effects of the extract of ACF in relieving the chemotherapy drug-induced nephritis or renal injury according to one embodiment of the present invention, wherein (a) is the Normal group, (b) the Cancer group, (c) the CGC group, (d) the CGCA group, and (e) the CA group; and

FIG. 13 shows the effects of the combinations of the extract of ACF and the chemotherapy drugs in the body weights of the experimental mice according to one embodiment of the present invention.

### DETAILED DESCRIPTION OF THE INVENTION

An embodiment will be used to demonstrate a method for fabricating an extract of ACF. Further, the methods and procedures of in vivo animal experiments are described in other embodiments for verifying the efficacies of the present invention.

### Embodiment I

### A Method for Fabricating an Extract of ACF and High-Performance Liquid Chromatography Analysis

1. The ACF used in the present invention meet the standard regulated by the draft of CNS15654: the fruiting body has a diameter of about 13 cm; the porosity featured by the surface of an ACF can be clearly observed under a dissecting microscope; each piece of the sections or slices of ACF has a dry weight of about 6-15 g.

### 2. Method for Fabricating an Extract of ACF

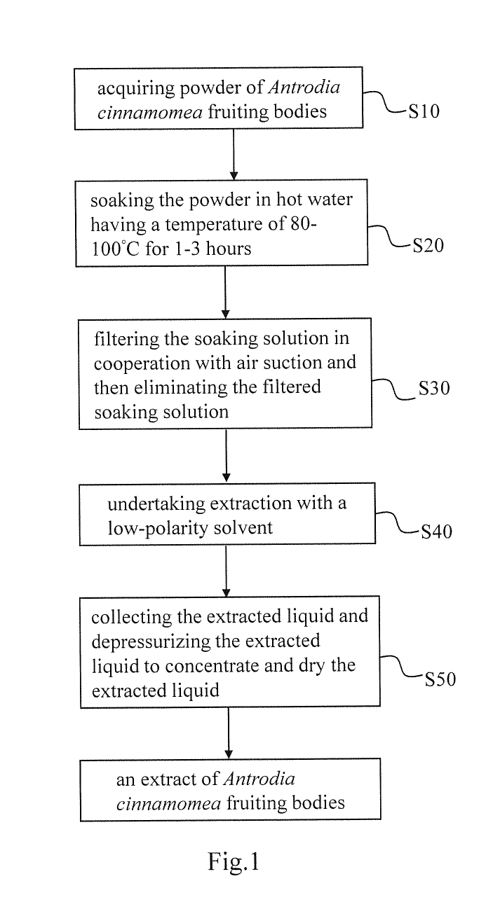

Refer to FIG. 1 for a flowchart of a method for fabricating an extract of ACF. In the embodiment shown in FIG. 1, the method for fabricating an extract of ACF comprises Steps S10-S50. In Step S10, acquire dried powder of ACF. In Step S20, undertake a first extraction step: soaking the powder in hot water, which has a volume of about 30 times the volume of the dried powder and a temperature of 80-100° C., for 1-3 hours. In Step S30, filtering the soaking solution with a No. 1 filter paper (ADVANTEC® #1) in cooperation with air suction and then eliminating the filtered soaking solution. In Step S40, the soaked powder was successively extracted with a low-polarity solvent, such as a 95% ethanol solution with 5 times the volume of dried powder weight, for 8 hours, and repeat it for three times. In Step S50, the resulting extracts were concentrated and dried under reduced pressure to obtain an extract of ACF (ACFE).
The low-polarity solvent is selected from a group including petroleum ether, normal hexane, ethyl acetate, acetone, ethanol, and combinations thereof. In one embodiment, the low-polarity solvent is a 25-100 wt % ethanol solution.

### 3. High-Performance Liquid Chromatography Analysis

A high-performance liquid chromatography device is used to analyze the components of the extract of ACF. The specification of the high-performance chromatography device is as follows: the high-performance chromatography device uses a pump of Spectra SYSTEM P1000, an automatic sampler of Spectra SYSTEM AS3000, a detector of Surveyor PDA Plus, a chromatographic column of Thermo, BDS HYPERSIL C18, 4.6*250 mm, a flow rate of 1.0 mL/min, and an ambient column temperature to detect a signal having a wavelength of 254 nm. The solvent system of the high-performance liquid chromatography analysis is shown in Table 1.

TABLE 1

| time | 0.1% FA | 0.1% FA/ACN |
|---|---|---|
| 0 | 70 | 30 |
| 40 | 50 | 50 |
| 60 | 50 | 50 |
| 80 | 0 | 100 |
| 120 | 0 | 100 |
| 122 | 70 | 30 |
| 130 | 70 | 30 |

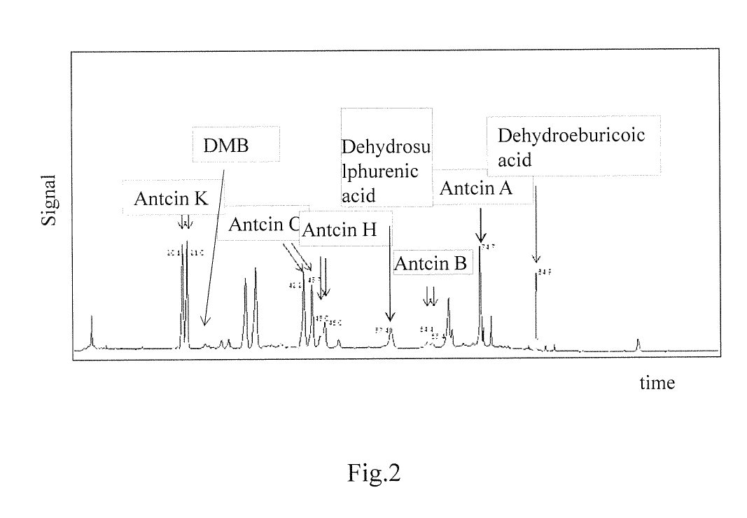

Refer to FIG. 2 for a spectrum obtained by the high-performance liquid chromatography (HPLC) analysis. The spectrum shows that the novel extract of ACF used by the present invention has 8 components identical to the characteristic components of the primitive ACF, including (R,S) antcin A, (R,S) Antcin B (zhankuic acid A), (R,S) antcin C, (R,S) antcin K, (R,S) antcin H, 4,7-dimethoxy-5-methyl-1,3-benzodioxole (DMB), dehydrosulphurenic acid, and dehydroeburicoic acid.

### Embodiment II

### In Vivo Experiments for Verifying the Effect of the Extract of ACF (Hereinafter Denoted by ACFE in Some Cases) Functioning as an Adjuvant to Chemotherapy Drugs (Cisplatin and Gemcitabine) in Lung Cancer Treatment

The experiment was conducted with the LLC (Lewis Lung Carcinoma) C57BL/6 lung cancer animal model. The animals used in the experiments are male C57BL/6 mice, 8 weeks old and weighing about 25 g. The LLC lung cancer cells were cultivated in a Dulbecco's Modified eagle medium (DMEM medium) containing 10% heat-inactivated fetal bovine serum (FBS) and 100 unit/mL of Penicillin-Streptomycin (P/S). The LLC cells were diluted with FBS to a concentration of 2×10⁷ cells/mL, and 0.1 mL of the cells, which have been adjusted to the desired concentration, was injected with soft needles to the tracheal tubes of the 8-week-old C57BL/6 mice. Then, the cells fall along the tracheal tube into the lung. Thus, each mouse was implanted with 2×10⁶ cells.

After the cancer cells have been implanted for one week, the mice are randomly arranged into 5 groups each containing 5 mice, including
1. A Normal group, whose mice were normal mice and fed with saline;
2. A Cancer group, whose mice were implanted with cancer cells and fed with saline;
3. A cancer+chemotherapy drug group (designated as the CGC group), whose mice were implanted with cancer cells and injected with chemotherapy drugs (cisplatin and gemcitabine);
4. A cancer+ACFE+chemotherapy drug group (designated as the CGCA group), whose mice were implanted with cancer cells, injected with clinical chemotherapy drugs (cisplatin and gemcitabine), and fed with ACFE by a dosage of 300 mg/kg/day; and
5. A cancer+ACFE group (designated as the CA group), whose mice are implanted with cancer cells and fed with ACFE by a dosage of 300 mg/kg/day.

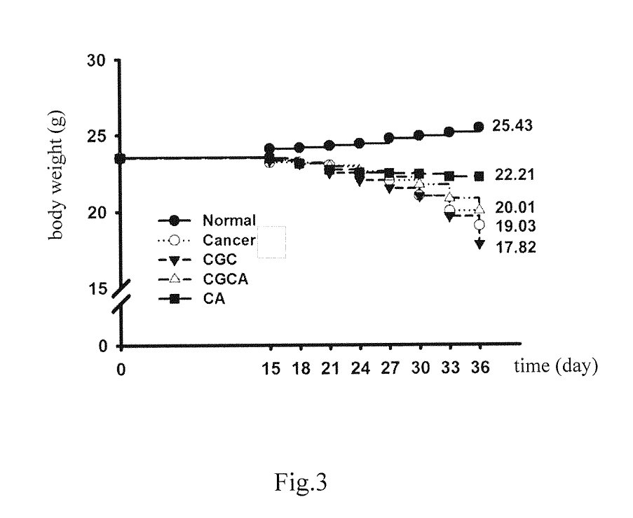

After the cancer has been (implanted to) induced in the mice for 10 days, the mice began to be fed with the tested drugs. The survival state and physiological state (body weight and food intake) of the mice were observed every day. The drug administration continued for 3 weeks (or 4 weeks according to the experiment design). Then, the mice were sacrificed, and the organs thereof were taken out, weighed, photographed, and analyzed pathologically. The residual organs were placed in 1.5 mL centrifugal tubes and then preserved in a refrigerator at a temperature of −80° C. for the succeeding analyses. The experimental results were shown in FIGS. 3-6.

Refer to FIG. 3 for the body weights of the mice. After 3 weeks of experiment, the Normal group had an average body weight of 25.43 g; the Cancer group had an average body weight of only 19.03 g, which was less than that of the Normal group by about 25.2%; the CGC group had an average body weight of only 17.82 g, which was the least one and less than that of the Cancer group by about 6.4%; the CGCA group, which were fed with ACFE, had an average body weight of as heavy as 20.01 g, which was more than that of the Cancer group by about 5.1%; the CA group, which were fed with ACFE, had an average body weight of as heavy as 22.21 g, which was more than that of the Cancer group by about 16.7%.

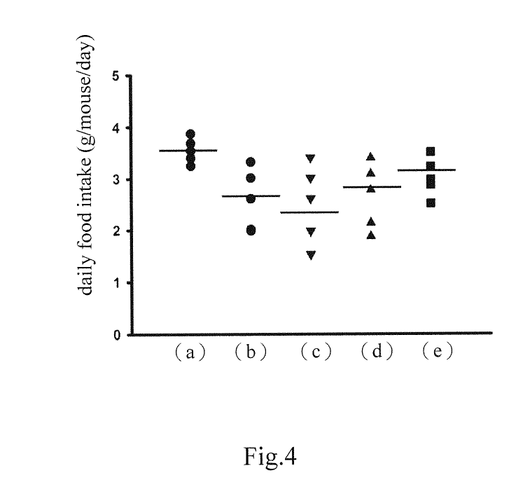

Refer to FIG. 4 for the food intakes of the mice. The experimental result revealed the following facts: the Normal group had a daily food intake of about 3.55±0.24 g; the Cancer group had a daily food intake of about 2.59±0.60 g, which were less than that of the Normal group by about 27%; the CGC group has a daily food intake of only 2.51±0.76, which were less than that of the Cancer group by about 3.0%; the CGCA group and the CA group, which were fed with ACFE, respectively had daily food intakes of about 2.65±0.64 g and 3.02±0.38 g, which were more than that of the Cancer group respectively by 2.3% and 16.6%. Therefore, the experiment proves that ACFE can obviously relieve the side-effect of decreased food intake caused by chemotherapy.

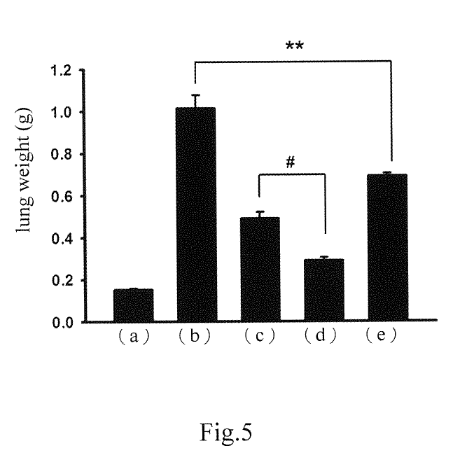

The appearance of the organs was observed to verify the effect of ACFE in decelerating the growth of lung cancer. The experimental result was shown that the growth of lung cancer was obviously slower in the CGCA group than in the CGC group. Refer to FIG. 5 for the lung weights of the mice. FIG. 5 revealed the following facts: the lung weight of the Normal group was about 0.16±0.01 g; the lung weight of the Cancer group was as heavy as 1.11±0.14 g, which was more than that of the Normal group by 593.8%; the lung weight of the CGC group was reduced to 0.49±0.07 g, which was less than that of the Cancer group by 55.9%; the lung weight of the CGCA group that were administered with chemotherapy drugs and ACFE was only about 0.34±0.05 g, which was less than that of the Cancer group by about 69.4% and had a statistically significant difference from that of the CGC group (p<0.01); the lung weight of the CA group that were fed with ACFE was less than that of the Cancer group by about 42.3% (p<0.005). Therefore, ACFE can significantly decrease the lung weight of lung-cancer mice.

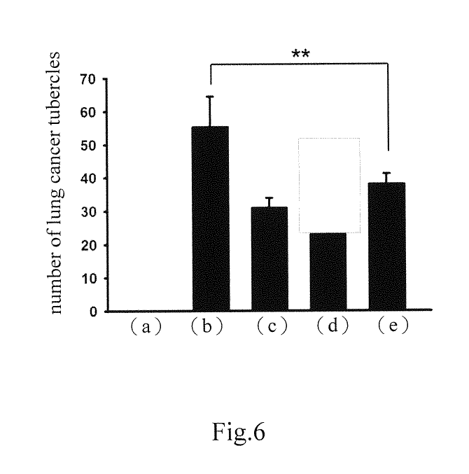

Refer to FIG. 6 for the numbers of lung cancer tubercles in the groups. The number of lung cancer tubercles of the Cancer group reached as many as 53.4±3.39. The number of lung cancer tubercles of the CA group that were fed with ACFE was 38.2±3.48, which was less than the lung cancer tubercles of the Cancer group by 28.5% and had a statistically significant difference from that of the Cancer group (p<0.005). The number of lung cancer tubercles of the CGC group merely using the chemotherapy drugs was 31.4±4.13. The number of lung cancer tubercles of the CGCA group was only 23.8±3.68 which was less than that of the Cancer group by 55.4% and also less than that of the CGC group.

### Embodiment III

### Effect of the Extract of ACF (ACFE) on Alopecia Caused by the Chemotherapy Drugs (Cisplatin and Gemcitabine)

Similarly to Embodiment II, drug administration was continuously applied to the experimental mice for 3 weeks and then stopped. The states of hair loss in the groups were photographed for observation. The experimental result was shown that alopecia in the CGC group was more obvious than in the Normal group. The CGC group had severe hair loss, which was significantly improved in the CGCA group that was fed with ACFE.

### Embodiment IV

### Effect of the Extract of Antrodia cinnamomea Fruiting Bodies (ACFE) on Muscle Degradation and Atrophy Caused by the Chemotherapy Drugs (Cisplatin and Gemcitabine)

Similarly to Embodiment II, drug administration was continuously applied to the experimental mice for 3 weeks. Then, the mice of the groups were sacrificed to obtain the thigh muscles thereof. The weight variations of gastrocnemius muscles and soleus muscles of the groups were statistically analyzed. The improvements of muscle atrophy were observed with a pathologic tissue section staining technology. The variations of the activities of the primary proteasomes in muscles were also analyzed, including chymotrypsin, trypsin, and caspase. The experimental result shows that the thigh muscles of the Cancer group and CGC group are obviously thinner than those of the Normal group and that muscle degradation and atrophy is significantly improved in the CGCA and CA groups that were fed with ACFE.

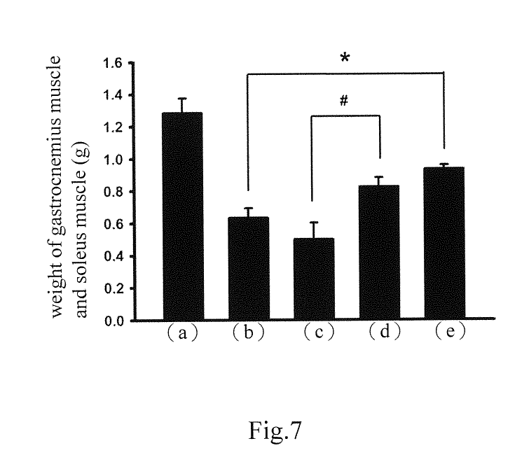

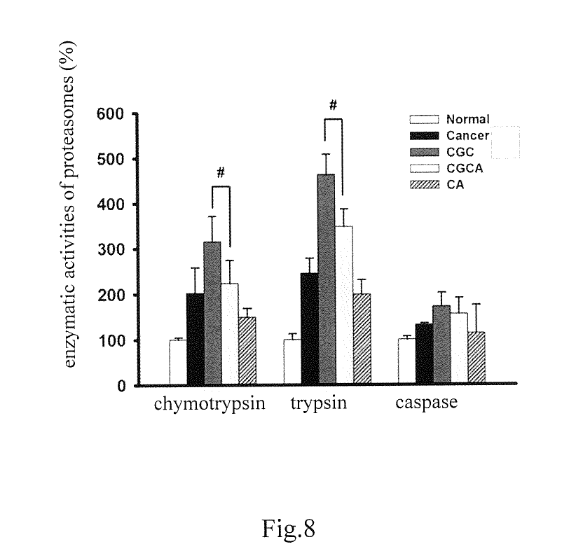

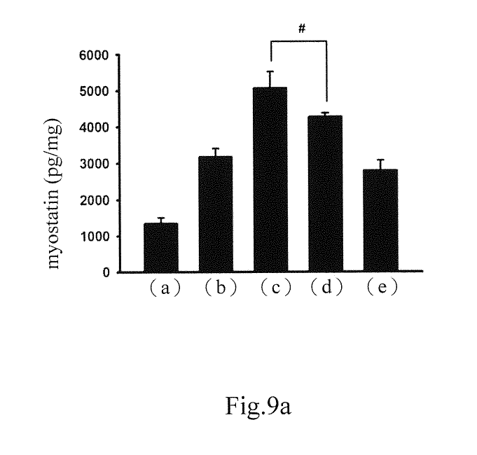

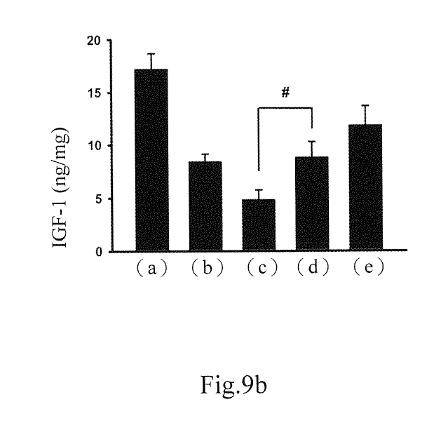

Refer to FIG. 7 for the weights of muscles. The experimental result shown that the weight of the gastrocnemius muscle and soleus muscle of the Normal group was 1.28±0.07 g and that the weights of the gastrocnemius muscles and soleus muscles of the Cancer group and the CGC group are respectively reduced to 0.6±0.03 g (lighter than that of the Normal group by 53.1%) and 0.57±0.05 g (lighter than that of the Normal group by 55.5%). Therefore, muscle atrophy in the Cancer group and the CGC group was more obvious than that in the other groups. However, the weights of the muscles of the CGCA and CA groups that were fed with ACFE respectively reached 0.81±0.11 g (heavier than that of the Cancer group by 35%) and 0.90±0.04 g (heavier than that of the Cancer group by 50%). In comparison with the CGC group without ACFE administration, the extract of Antrodia cinnamomea fruiting bodies (ACFE) can significantly improve muscle degradation and atrophy in the CGCA and CA groups (p<0.01). The H&E staining of pathologic tissue section also was shown that the extract of Antrodia cinnamomea fruiting bodies (ACFE) can significantly improve muscle degradation in the CGCA and CA groups that were fed with ACFE. Refer to FIG. 8 for the enzymatic expression of the primary proteasomes in muscles and also revealed the following facts: the CGC group had the highest expression of chymotrypsin and trypsin in muscle among all the groups; the expression of chymotrypsin and trypsin were obviously lowered in the CGCA group that were fed with ACFE (p<0.01); the CGC group had the highest expression of caspase that executes apoptosis. Administration of ACFE assisted in lowering the proteasome expression, which were increased by chemotherapy drugs. The possible mechanism of lowering the proteasome expression assumed that ACFE inhibited the expression of myostatin and encouraged the expression of IGF-1 (refer to FIG. 9a and FIG. 9b).

### Embodiment V

### Effect of the Extract of Antrodia cinnamomea Fruiting Bodies (ACFE) in Inhibiting Blood Inflammatory Factors Increasing Caused by the Chemotherapy Drugs (Cisplatin and Gemcitabine)

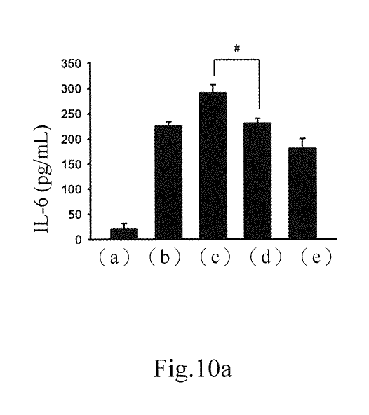

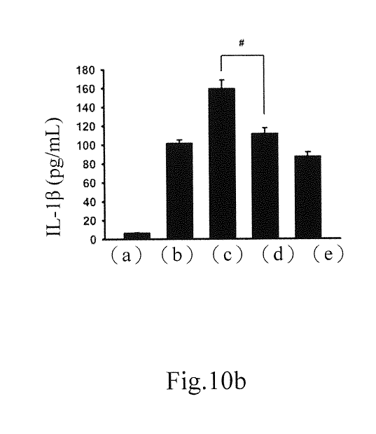

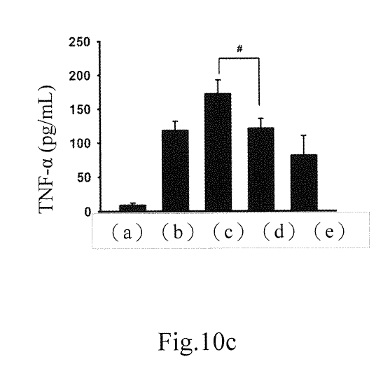

Similarly to Embodiment II, different treatments were continuously applied to the LLC (Lewis Lung Carcinoma) C57BL/6 lung cancer animal model for 3 weeks (or 4 weeks according to the experiment design). Then, the mice were sacrificed to sample the blood thereof. The blood was further to analyzed concentration of interleukin-6 (IL-6), interleukin-1β (IL-1β), and the tumor necrosis factor α (TNF-α). FIGS. 10a-10c revealed the following facts: the CGC group shown the highest concentrations of the blood inflammatory factors IL-6, IL-1β and TNF-α among all the groups; the concentrations of the blood inflammatory factors IL-6, IL-1β and TNF-α were significantly reduced in the CGCA group that were fed with ACFE (p<0.01) (refer to FIGS. 10a-10c).

### Embodiment VI

### Effect of the Extract of Antrodia cinnamomea Fruiting Bodies (ACFE) in Improving Gastrointestinal Tract Lesion Caused by the Chemotherapy Drugs (Cisplatin and Gemcitabine)

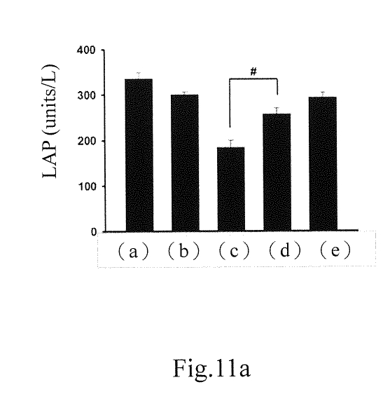

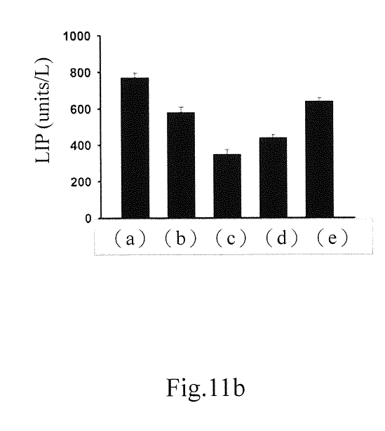

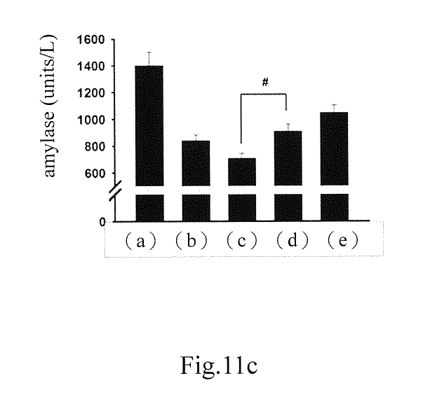

Similarly to Embodiment II, different treatments were continuously applied to LLC (Lewis Lung Carcinoma) C57BL/6 lung cancer animal model for 3 weeks (or 4 weeks according to the experiment design). Then, the mice were sacrificed and further conducted the e small intestine villus pathological sections. The small intestine villus sections were observed the injury severity of villus lesion after H&E staining. The experimental result shown that ACFE can obviously decrease the side-effects of small intestine villus lesion and gastric ulcer caused by the chemotherapy drugs (cisplatin and gemcitabine) and can significantly increase the enzymatic expression of leucine aminopeptidase (LAP), lipase (LIP) and amylase in small intestines, as shown in FIGS. 11a-11c.

### Embodiment VII

### Effect of the Extract of Antrodia cinnamomea Fruiting Bodies (ACFE) in Improving Nephritis and Renal Injury Caused by the Chemotherapy Drugs (Cisplatin and Gemcitabine)

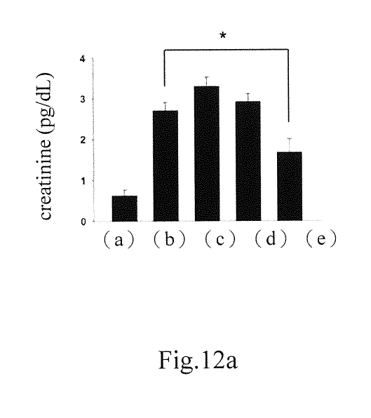

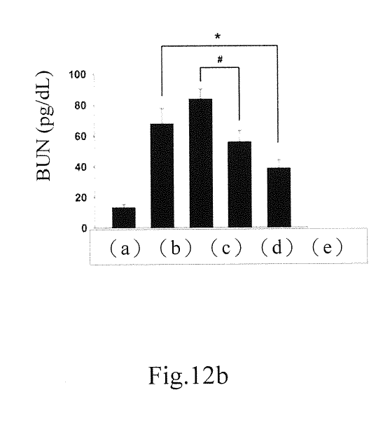

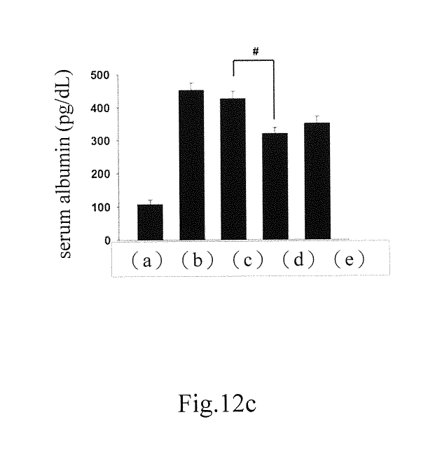

Similarly to Embodiment II, different treatments were continuously applied to the LLC (Lewis Lung Carcinoma) C57BL/6 lung cancer animal model for 3 weeks (or 4 weeks according to the experiment design). Then, the mice of the groups were sacrificed to sample the blood thereof. The blood was further analyzed the concentrations of creatinine, blood urea nitrogen (BUN), and serum albumin in the blood of all the groups. The experimental result show that ACFE can obviously lower the nephritis indexes creatinine, blood urea nitrogen (BUN), and serum albumin in the blood of the experimental mice administered with the chemotherapy drugs (cisplatin and gemcitabine), as shown in FIGS. 12a-12c.

### Embodiment VIII

### The Dosage Determination Experiment for Applying the Extract of Antrodia Cinnamomea Fruiting Bodies (ACFE) as an Adjuvant Chemotherapy Drugs (Cisplatin and Gemcitabine) for Lung Cancer Treatment

A subcutaneous implantation model (SC model) was used to evaluate the adjuvant chemotherapy effect of ACFE on tumor growth.

The LLC mouse lung cancer cells were cultivated in a Dulbecco's Modified eagle medium (DMEM medium) containing 10% heat-inactivated fetal bovine serum (FBS) and 100 unit/mL of Penicillin-Streptomycin (P/S), which was placed in a thermostatic incubator having 5% CO₂, a temperature of 37° C. and a humidity of 90%. The cells were maintained at a density of 2×10⁵-1×10⁶ (the culture dish is 80-90% filled). The culture medium was refreshed periodically every 2 or 3 days.

The LLC cells were taken out with 0.05% Trypsin-EDTA and diluted with FBS to a concentration of 1×10⁷ cells/mL, and 0.1 mL of the cells, which had been modified to the desired concentration, was subcutaneously injected with insulin-syringes into the right backs of the 8-week-old C57BL/6 mice. Thus, each mouse was implanted with 1×10⁶ cells.

After the cancer cells had been implanted for one week, the mice were randomly arranged into 5 groups each containing 5 mice, including
1. A Cancer group, whose mice were implanted with cancer cells;
2. A cancer+chemotherapy drug group (designated as the CGC group), whose mice were implanted with cancer cells and injected with clinical chemotherapy drugs cisplatin and gemcitabine;
3. A cancer+ACFE+chemotherapy drug group (designated as the CGCA300 group), whose mice were implanted with cancer cells, injected with clinical chemotherapy drugs cisplatin and gemcitabine, and fed with ACFE at a dosage of 300 mg/kg/day;
4. A cancer+ACFE+chemotherapy drug group (designated as the CGCA150 group), whose mice were implanted with cancer cells, injected with clinical chemotherapy drugs cisplatin and gemcitabine, and fed with ACFE at a dosage of 150 mg/kg/day; and
5. A cancer+ACFE+chemotherapy drug group (designated as the CGCA75 group), whose mice were implanted with cancer cells, injected with clinical chemotherapy drugs cisplatin and gemcitabine, and fed with ACFE at a dosage of 75 mg/kg/day.

After the cancer had been induced in the mice for 10 days, the mice began to be fed with the tested drugs. Each 3 days, the growth states of the cancers were observed, and the sizes (volumes) of the cancers were calculated according to an equation: 0.53×length×width². The drug administration continued for 2 weeks, and the mice were sacrificed next day. During the experiment, the cancer sizes, hair losses, body weight variations and food intakes were observed and evaluated. The data was analyzed and plotted with SigmaPlot software and the data was expressed in mean+SEM.

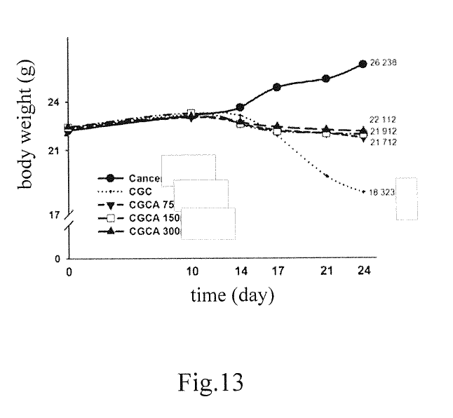

Refer to FIG. 13 for body weight variations of the experimental mice. The body weight of the Cancer group was as heavy as 26.2 g. The body weight of the CGC group was only 18.3 g (lighter than that of the Cancer group by 30%). The CGCA75 group, CGCA150 group and CGCA300 group, which were separately administered with different dosages of ACFE, respectively weigh 21.7 g, 21.9 g and 22.1 g, which were respectively heavier than that of the CGC group by 18.6%, 19.6% and 20.8%. The experimental result indicated that 75 mg/kg/day or higher dosage of ACFE can significantly improve the weight loss caused by the chemotherapy drugs (cisplatin and gemcitabine). The further results were also shown that over 75 mg/kg/day of ACFE can significantly improve hair graying and hair loss of test animals caused by the chemotherapy drugs (cisplatin and gemcitabine).

On the growth inhibition experiment of ACFE for lung cancer, the test results revealed the following facts: the Cancer group had the greatest cancer weight, which was significantly different from that of the CGC group (p<0.001); the CGCA300 group and the CGCA150 were significantly different from the CGC group in cancer weight (p<0.001). The experimental result proved that ACFE can obviously enhance the lung cancer treatment effect of the chemotherapy drugs (cisplatin and gemcitabine).

Therefore, the ACFE can effectively exhibit the lung cancer-inhibiting effect combined the chemotherapy drugs (cisplatin and gemcitabine) and can also effectively relieve alopecia, degradation and atrophy of muscle, gastrointestinal lesion, nephritis caused by the chemotherapy drugs. The ACFE can also be fabricated into a pharmaceutical composition in various drug forms to function as an adjuvant chemotherapy drugs.

The pharmaceutical composition of the present invention at least comprises an effective dosage of ACFE and a possible and acceptable pharmaceutical carrier. The pharmaceutical composition of the present invention can be fabricated into oral drugs in form of tablets, capsules, dropping pills, emulsions, electuaries, aqueous suspensions, and dispersion liquids. However, the present invention does not limit that the pharmaceutical composition must be fabricated into one of the above-mentioned forms. The pharmaceutical composition of the present invention can function as an adjuvant drug to enhance the cancer treatment effect of chemotherapy drugs and relieve the syndromes of cancers and the side-effects of chemotherapy drugs. In one embodiment, the dosage of the ACFE is 75-300 mg/kg. Considering the ACFE has a water content of about 25%, the preferred effective dosage for the in vivo mice model is 56-225 mg/kg.

In conclusion, the present invention discloses a pharmaceutical composition adjuvant to chemotherapy drugs and applications thereof. The pharmaceutical composition of the present invention can be administered together with chemotherapy drugs to enhance the treatment effect of the chemotherapy drugs and relieve the syndromes of cancers and the side-effects caused by the chemotherapy drugs. The present invention will provide practical assistance to clinical cancer treatment.

The embodiments described above are only to exemplify the present invention but not to limit the scope of the present invention. Any equivalent modification or variation according to the characteristic or spirit of the present invention is to be also included within the scope of the present invention.

What is claimed is:

1. A pharmaceutical composition adjuvant to chemotherapy drugs, comprising
an effective dosage of an extract of Antrodia cinnamomea fruiting bodies; and
an acceptable pharmaceutical carrier,
wherein said extract of Antrodia cinnamomea fruiting bodies is an adjuvant drug of said chemotherapy drugs to enhance cancer treatment effect of said chemotherapy drugs and relieve a syndrome of a cancer and a side-effect caused by said chemotherapy drugs, and wherein said chemotherapy drugs are cisplatin and gemcitabine.

2. The pharmaceutical composition according to claim 1, wherein said extract of Antrodia cinnamomea fruiting bodies includes antcin K, antcin C, antcin H, and derivatives thereof.

3. The pharmaceutical composition according to claim 1, wherein said extract of Antrodia cinnamomea fruiting bodies includes dehydrosulphurenic acid, dehydroeburicoic acid, and derivatives thereof.

4. The pharmaceutical composition according to claim 1, wherein said extract of Antrodia cinnamomea fruiting bodies includes antcin B, antcin A, and derivatives thereof.

5. The pharmaceutical composition according to claim 1, wherein said extract of Antrodia cinnamomea fruiting bodies includes 4,7-dimethoxy-5-methyl-1,3-benzodioxole (DMB), and derivatives thereof.

6. The pharmaceutical composition according to claim 1, which is fabricated into oral medicine in form of a tablet, a capsule, a dropping pill, an emulsion, an electuary, an aqueous suspension, or a dispersion liquid.

7. The pharmaceutical composition according to claim 1, wherein an effective dosage of said extract of Antrodia cinnamomea fruiting bodies for an in vivo mice model is 56-225 mg/kg.

8. The pharmaceutical composition according to claim 1, wherein said extract of Antrodia cinnamomea fruiting bodies is fabricated with steps:
soaking Antrodia cinnamomea fruiting bodies in hot water at a temperature of 80-100° C. for 1-3 hours to obtain a soaking solution;
filtering said soaking solution and then eliminating the filtered soaking solution to obtain a soaked powder;
undertaking extraction with a low-polarity solvent to obtain said extract of Antrodia cinnamomea fruiting bodies from said soaked powder.

9. The pharmaceutical composition according to claim 8, wherein said low-polarity solvent is selected from a group including petroleum ether, normal hexane, ethyl acetate, acetone, ethanol, and combinations thereof.

10. The pharmaceutical composition according to claim 1, wherein said cancer is a lung cancer.

11. The pharmaceutical composition according to claim 1, wherein said side-effect caused by said chemotherapy drugs includes at least one of hair loss, degradation and atrophy of muscle, gastrointestinal lesion, nephritis, or renal injury.

12. An application of a pharmaceutical composition, wherein said pharmaceutical composition comprises an effective dosage of an extract of Antrodia cinnamomea fruiting bodies and an acceptable pharmaceutical carrier, and wherein said application is to function as an adjuvant drug of chemotherapy drugs to enhance cancer treatment effect of said chemotherapy drugs and relieve a syndrome of a cancer and a side-effect caused by said chemotherapy drugs, and wherein said chemotherapy drugs are cisplatin and gemcitabine.

13. The application according to claim 12, wherein said cancer is a lung cancer.

14. The application according to claim 12, wherein said side-effect caused by said chemotherapy drugs includes at least one of hair loss, degradation and atrophy of muscle, gastrointestinal lesion, nephritis, or renal injury.

15. The application according to claim 12, wherein an effective dosage of said extract of Antrodia cinnamomea fruiting bodies for an in vivo mice model is 56-225 mg/kg.

16. The application according to claim 12, wherein said extract of Antrodia cinnamomea fruiting bodies includes antcin K, antcin C, antcin H, and derivatives thereof.

17. The application according to claim 12, wherein said extract of Antrodia cinnamomea fruiting bodies includes dehydrosulphurenic acid, dehydroeburicoic acid, and derivatives thereof.

18. The application according to claim 12, wherein said extract of Antrodia cinnamomea fruiting bodies includes antcin B, antcin A, and derivatives thereof.

19. The application according to claim 12, wherein said extract of Antrodia cinnamomea fruiting bodies includes 4,7-dimethoxy-5-methyl-1,3-benzodioxole (DMB), and derivatives thereof.

20. The application according to claim 12, wherein said pharmaceutical composition is fabricated into oral medicine in form of a tablet, a capsule, a dropping pill, an emulsion, an electuary, an aqueous suspension, or a dispersion liquid.

21. The application according to claim 12, wherein said extract of Antrodia cinnamomea fruiting bodies is fabricated with steps:
soaking Antrodia cinnamomea fruiting bodies in hot water at a temperature of 80-100° C. for 1-3 hours to obtain a soaking solution;
filtering said soaking solution and then eliminating the filtered soaking solution to obtain a soaked powder;
undertaking extraction with a low-polarity solvent to obtain said extract of Antrodia cinnamomea fruiting bodies from said soaked powder.

22. The application according to claim 21, wherein said low-polarity solvent is selected from a group including petroleum ether, normal hexane, ethyl acetate, acetone, ethanol, and combinations thereof.
